(19) United States
(12) Patent Application Publication
(10) Pub. No.: US 2023/0260773 A1
(43) Pub. Date: Aug. 17, 2023

(54) METHOD FOR CONSTRUCTING CORRELATION MODEL BETWEEN RELATIVE BIOAVAILABILITY AND BIOACCESSIBILITY OF CADMIUM IN EARTHWORM

(71) Applicants: National Institutes for Food and Drug Control, Beijing (CN); Wang Jing Hospital of CACMS (Orthopedics Research Institute of CACMS), Beijing (CN); Tongliao Market Inspection and Testing Center, Tongliao City (CN)

(72) Inventors: Shuangcheng Ma, Beijing (CN); Tiantian Zuo, Beijing (CN); Jia Zhu, Beijing (CN); Qinghui Song, Beijing (CN); Wanqiang Zhang, Beijing (CN); Jishuang Wang, Beijing (CN); Haiyan Wang, Beijing (CN); Lei Sun, Beijing (CN); Hongyu Jin, Beijing (CN); Qi Wang, Beijing (CN); Jianbo Yang, Beijing (CN); Dejuan Kong, Tongliao City (CN); Shuai Kang, Beijing (CN); Jiandong Yu, Beijing (CN)

(73) Assignees: National Institutes for Food and Drug Control, Beijing (CN); Wang Jing Hospital of CACMS (Orthopedics Research Institute of CACMS), Beijing (CN); Tongliao Market Inspection and Testing Center, Tongliao City (CN)

(21) Appl. No.: 17/804,321

(22) Filed: May 27, 2022

(30) Foreign Application Priority Data
Feb. 16, 2022 (CN) 202210139716.4

(51) Int. Cl. H01J 49/10; H01J 49/00

(52) U.S. Cl. CPC H01J 49/105; H01J 49/0036

(57) ABSTRACT

The present disclosure provides a method for constructing a correlation model between relative bioavailability and bioaccessibility of cadmium in earthworm, belonging to the technical field of cadmium detection in earthworms. The method includes the following steps: determination of relative bioavailability of cadmium in earthworms, determination of bioaccessibility of cadmium in the earthworm based on an in vitro physiologically-based extraction test (PBET) method, and construction of a correlation model between the relative bioavailability and the bioaccessibility of cadmium in the earthworm. The method enables to directly put the bioaccessibility obtained by the in vitro method into a regression equation to obtain the bioavailability of cadmium in subsequent studies. The method eliminates a need for animal experiments in follow-up researches, saves resources to the greatest extent, and provides a new idea for objectively and scientifically evaluating the health risk of cadmium in the earthworm to human bodies.

### CROSS REFERENCE TO RELATED APPLICATION

This application claims priority to Chinese Patent Application No. 202210139716.4, filed Feb. 16, 2022, the content of which is incorporated by reference in its entirety.

### TECHNICAL FIELD

The present disclosure relates to the technical field of cadmium detection in earthworm, in particular to a method for constructing a correlation model between relative bioavailability and bioaccessibility of cadmium in earthworm.

### BACKGROUND ART

More and more attention is paid to the safety of traditional Chinese medicines. Studies have found that heavy metal pollution is one of the main reasons for health hazards of the Chinese medicinal materials. Air and soil pollution, and processing and transportation of the Chinese medicinal materials will aggravate the pollution of heavy metals in the Chinese medicinal herbs. A long-term use of Chinese medicinal materials contaminated by heavy metals may cause accumulation of the heavy metals in the human body, resulting in a series of health hazards. For example, earthworm is a commonly used animal medicinal material in China. The “Shen Nong's herbal classic” records that “earthworms have cold and salty property and flavor, and play a role in the lung, kidney, and liver meridians”. The earthworm is generally used for treating symptoms such as high fever, dizziness, epilepsy convulsions, lung heat, asthma and cough. The earthworms prefer to live in soil, and heavy metals in the environment and soil are easy to accumulate in the earthworms, eventually causing serious physiological toxicity to the human body through the food chain.

Cadmium is a heavy metal and can cause bone damages, and long-term accumulation of cadmium in the body will also lead to a certain degree of harms to the kidney, cardiovascular system and other systems. Therefore, it is necessary to conduct a scientific and reasonable assessment of a risk of cadmium. In recent years, risk researches on heavy metal pollutants in food and drinking water have been greatly developed, and risk assessments of exogenous harmful residues in Chinese medicinal materials are also gradually emerging. However, assessment methods for the exogenous harmful residues mainly include total residues based on heavy metals. Most current calculation methods for heavy metal exposure are based on contaminant intake (external exposure) rather than in vivo exposure closely related to contaminant toxicity. However, not all substances are released from the matrix during digestion and absorbed by the human body, resulting in a bioavailability of less than 100%. The evaluation of health risks on the heavy metals in Chinese medicinal materials using a total amount of the heavy metals in Chinese medicinal materials tends to overestimate the harm of heavy metals to the human body, leading to unnecessary governmental intervention and waste of resources. Therefore, it is of great significance for accurately evaluating the health risks of cadmium in the earthworm to the human body by researching the bioavailability of cadmium in the human body.

### SUMMARY

The objective of the present disclosure is to provide a method for constructing a correlation model between relative bioavailability and bioaccessibility of cadmium in earthworm. The method enables to directly put the bioaccessibility obtained by the in vitro method into a regression equation to obtain the bioavailability of cadmium in subsequent studies. The method eliminates a need for follow-up researches and animal experiments, saves resources to the greatest extent, and provides a new idea for objectively and scientifically evaluating the health risk of cadmium in the earthworm to human bodies.

To achieve the above objective, the present disclosure provides the following technical solutions.

The present disclosure provides a method for constructing a correlation model between relative bioavailability and bioaccessibility of cadmium in earthworm, including the following steps:

grouping mice, conducting successive administration for 6 days to 8 days, and sacrificing the mice; determining a cadmium content in liver, kidney, brain, and femur tissues of the mice, and calculating relative bioavailability of cadmium in earthworm;

simulating gastrointestinal digestion by a physiologically-based extraction test (PBET) method and extracting digestive juices, and determining a residual amount of cadmium in gastric juice and intestinal juice separately to obtain bioaccessibility of cadmium in the earthworm; and

constructing a correlation model according to the relative bioavailability and the bioaccessibility of cadmium in the earthworm to obtain a regression equation.

Preferably, the grouping may be conducted specifically as follows: the mice fed with normal saline are used as a blank control group, the mice fed with cadmium are used as a positive control group, and the mice fed with earthworm are used as a earthworm group; and the positive control group may be divided into a low-concentration positive control group, a medium-concentration positive control group, and a high-concentration positive control group.

Preferably, the low-concentration positive control group may have a cadmium concentration of 3.5 mg/kg to 4.5 mg/kg; the medium-concentration positive control group may have a cadmium concentration of 18 mg/kg to 22 mg/kg; the high-concentration positive control group may have a cadmium concentration of 95 mg/kg to 105 mg/kg; and the earthworm group may have a earthworm concentration of 1.128 mg/kg to 7.401 mg/kg.

Preferably, a calculation formula of the relative bioavailability may be as follows:

$$Relative bioavailability = \frac{{( Liver + kidney + brain + femur )}_{earthworm}}{Earthworm concentration} \times \frac{Positive control group concentration}{{( Liver + kidney + brain + femur )}_{positive}}$$

where, (liver 30 kidney+brain+femur)_{Earthworm} is the sum of cadmium content in the liver, kidney, brain and femur tissues of the mice determined 6 days to 8 days after administration of the Earthworm group, (liver+kidney+brain+femur)ₚₒₛᵢₜᵢᵥₑ is the sum of cadmium content in the liver, kidney, brain and femur tissues of the mice determined 6 days to 8 days after administration of the low-concentration positive control group, the medium-concentration positive control group, and the high-concentration positive control group.

Preferably, cadmium content in the tissues may be determined by an inductively coupled plasma-mass spectrometry (ICP-MS) method; and the residual amount of cadmium in the gastric juice and the intestinal juice may be determined by the ICP-MS method.

Preferably, the administration may be conducted by gavage.

Preferably, working parameters of the ICP-MS method may be as follows:

| | | | | Working | |
|---|---|---|---|---|---|
| Item | Working | | Item | parameter | |
| Plasma gas flow | 15.0 | L/min | Peristaltic pump | 0.20 | r/s |
| Sampling depth | 10 | mm | Number of | 1 | |
| Nebulization | 2° | C. | Auxiliary gas flow | 0.8 | L/min |
| chamber | | | | | |
| He gas flow | 5 | mL/min | Carrier gas flow | 0.8 | L/min |

| | | | | |
|---|---|---|---|---|
| Radio-frequency | 1550 | W | Data | Peak |
| power | | | sampling | hopping |
| | | | mode | sampling |

| | | | |
|---|---|---|---|
| Determination | 6 | Number | 100 |
| point/peak | | of scans | |
| Number of | 3 | | |
| repetitions | | | |

Preferably, the regression equation may be as follow: relative bioavailability=1.09×bioaccessibility+6.97.

Compared with the prior art, the present disclosure has the following beneficial effects:

The present disclosure provides a method for constructing a correlation model between relative bioavailability and bioaccessibility of cadmium in earthworm. The present disclosure constructs for the first time an in vivo and in vitro correlation model of cadmium in earthworm medicinal materials. The method can scientifically, objectively, and accurately correct results of cadmium bioaccessibility in the earthworm obtained by the in vitro PBET method. In follow-up studies, the in vitro method can be directly substituted into the regression equation to obtain bioavailability results of cadmium in the earthworm, and the results can be used in subsequent risk assessments. The method eliminates a need for animal experiments in follow-up researches. Compared with complex animal experiments of a long period and high cost, the method is simpler and faster, and saves resources to the greatest extent. The present disclosure has important significance for accurately evaluating health risks of cadmium in the earthworm to the human body, and provides a new idea for objectively and scientifically evaluating the health risks of cadmium in the earthworm to the human body. The present disclosure also provides a scientific basis for improving a safety technical standard system for harmful residues of the earthworm, effectively responding to occurrence of drug injury events, and ensuring the people's medication safety.

### BRIEF DESCRIPTION OF THE DRAWINGS

FIG. 1 shows results of relative bioavailability of cadmium in 10 batches of earthworm; and

FIG. 2 shows a correlation between bioavailability and bioaccessibility of cadmium in earthworm.

### DETAILED DESCRIPTION OF THE EMBODIMENTS

The present disclosure provides a method for constructing a correlation model between relative bioavailability and bioaccessibility of cadmium in earthworm, including the following steps:

grouping mice, conducting successive administration for 6 days to 8 days, and sacrificing the mice; determining a cadmium content in liver, kidney, brain, and femur tissues of the mice, and calculating relative bioavailability of cadmium in earthworm;

simulating gastrointestinal digestion by a physiologically-based extraction test (PBET) method and extracting digestive juice, and determining a residual amount of cadmium in gastric juice and intestinal juice separately to obtain bioaccessibility of cadmium in the earthworm; and

constructing a correlation model according to the relative bioavailability and the bioaccessibility of cadmium in the earthworm to obtain a regression equation.

In the present disclosure, the mice are grouped, administered continuously for 6 days to 8 days, and then sacrificed. Preferably, the grouping is conducted specifically as follows: the mice fed with normal saline are used as a blank control group, the mice fed with cadmium are used as a positive control group, and the mice fed with the earthworm are used as an earthworm group; and the positive control group is divided into a low-concentration positive control group, a medium-concentration positive control group, and a high-concentration positive control group. The low-concentration positive control group has a cadmium concentration of preferably 3.5 mg/kg to 4.5 mg/kg; the medium-concentration positive control group has a cadmium concentration of preferably 18 mg/kg to 22 mg/kg; the high-concentration positive control group has a cadmium concentration of preferably 95 mg/kg to 105 mg/kg; and the earthworm group has an earthworm concentration of preferably 1.128 mg/kg to 7.401 mg/kg, the administration is conducted preferably by gavage. More preferably, the grouping is conducted specifically as follows:

1) control group: 6 mice are subjected to administration of a normal saline at 1 mL/mice by gavage, once a day;

2) low-concentration positive control group: 6 mice are subjected to administration at 1 mL/mice by gavage with a Cd concentration of 4 mg/kg, once a day;

3) medium-concentration positive control group: 6 mice are subjected to administration at 1 mL/mice by gavage with a Cd concentration of 20 mg/kg, once a day;

4) high-concentration positive control group: 6 mice are subjected to administration at 1 mL/mice by gavage with a Cd concentration of 100 mg/kg, once a day; and

5) earthworm group: 6 mice are subjected to administration at 1 mL/mice by gavage with an earthworm concentration of 5 mg/kg, once a day.

In the present disclosure, cadmium content in liver, kidney, brain, and femur tissues of the mice is determined, and relative bioavailability of cadmium in earthworm is calculated. Each tissue of the liver, kidney, brain, and femur is weighed, freeze-dried, and digested before determining cadmium content in the tissues. There is no special limitation on the method of freeze-drying and digestion, and methods known in the art can be used. The cadmium content in the tissues is determined preferably by an ICP-MS method. After the determination of cadmium content, relative bioavailability is calculated, where the calculation formula of the relative bioavailability is preferably as follows:

$$Relative bioavailability = \frac{{( Liver + kidney + brain + femur )}_{Earthworm}}{Earthworm concentration} \times \frac{Positive control group concentration}{{( Liver + kidney + brain + femur )}_{positive}} ;$$

where,

(liver+kidney+brain+femur)_{Earthworm} is the sum of cadmium content in the liver, kidney, brain and femur tissues of the mice determined 6 days to 8 days after administration of the earthworm group (unit: μg/g dry weight), (liver+kidney+brain+femur)ₚₒₛᵢₜᵢᵥₑ is a sum of cadmium content in the liver, kidney, brain and femur tissues of the mice determined 6 days to 8 days after administration of the low-concentration positive control group, the medium-concentration positive control group, and the high-concentration positive control group (unit: μg/g dry weight); and the positive control group concentration or the earthworm concentration is calculated in μg/g body weight A combined exposure method in which liver, kidney, brain, and femur are used as an action target is used, such that the relative bioavailability determined is more stable.

In the present disclosure, gastrointestinal digestion is simulated by a PBET method and digestive juices are extracted, and a residual amount of cadmium in gastric juice and intestinal juice is determined to obtain bioaccessibility of cadmium in the earthworm. There is no special limitation on specific steps for simulating the gastrointestinal digestion by the PBET method and extracting the digestive juices, and methods known in the art can be used. The residual amount of cadmium in the gastric juice and the intestinal juice is preferably determined by the ICP-MS method.

The working parameters of the ICP-MS method are preferably as follows:

| | | | | Working | |
|---|---|---|---|---|---|
| Item | Working | | Item | parameter | |
| Plasma gas flow | 15.0 | L/min | Peristaltic pump | 0.20 | r/s |
| Sampling depth | 10 | mm | Number of | 1 | |
| | | | repetitions | | |
| Nebulization | 2° | C. | Auxiliary | 0.8 | L/min |
| chamber | | | gas flow | | |
| He gas flow | 5 | mL/min | Carrier gas flow | 0.8 | L/min |

| | | | | |
|---|---|---|---|---|
| Radio-frequency | 1550 | W | Data | Peak |
| power | | | sampling | hopping |
| | | | mode | sampling |

| | | | |
|---|---|---|---|
| Determination | 6 | Number | 100 |
| point/peak | | of scans | |
| Number of | 3 | | |
| repetitions | | | |

The cadmium content in earthworm was determined using the ICP-MS working parameters, and a standard curve was drawn. According to an analysis of a fitting degree of the curve, a correlation coefficient of cadmium was determined to be 0.997, meaning a desirable linear relationship; and suitability of the method was confirmed using accompanying quality control, and results were in line with residue testing requirements.

In the present disclosure, a correlation model is constructed according to the relative bioavailability and the bioaccessibility of cadmium in the earthworm to obtain a regression equation. The regression equation is as follow: relative bioavailability=1.09×bioaccessibility+6.97, where r²=0.84.

In the present disclosure, unless otherwise specified, all raw material components are commercially available products well known to those skilled in the art.

The technical solutions are clearly and completely described below in conjunction with examples of the present disclosure. It is clear that the described examples are merely a part, rather than all of the examples of the present disclosure. All other examples obtained by a person of ordinary skill in the art based on the examples of the present disclosure without creative efforts shall fall within the protection scope of the present disclosure.

### EXAMPLE 1

A method for constructing a correlation model between relative bioavailability and bioaccessibility of cadmium in earthworm included the following steps:

(1) Determination of Relative Bioavailability of Cadmium in Earthworm

After adaptive feeding, 18 g to 22 g of female mice were randomly grouped (6 mice/group), and weighed after fasting overnight; 10 batches of earthworm groups were given multiple times of intragastric administration of earthworm; high-concentration, medium-concentration and low-concentration positive control groups were administered with corresponding concentrations of cadmium by gavage; meanwhile, mice administered with normal saline by gavage were used as a blank control group. The grouping and administration were conducted as follows:

1) control group: 6 mice were subjected to administration of normal saline at 1 mL/mice by gavage;, once a day;

2) low-concentration positive control group: 6 mice were subjected to administration at 1 mL/mice by gavage with a Cd concentration of 4 mg/kg, once a day;

3) medium-concentration positive control group: 6 mice were subjected to administration at 1 mL/mice by gavage with a Cd concentration of 20 mg/kg, once a day;

4) high-concentration positive control group: 6 mice were subjected to administration at 1 mL/mice by gavage with a Cd concentration of 100 mg/kg, once a day; and

5) earthworm group: 6 mice were subjected to administration at 1 mL/mice by gavage with a earthworm concentration of 5 mg/kg, once a day.

After 7 days of continuous administration, the mice were sacrificed, and liver, kidney, brain, and femur tissues of the mice were taken out, weighed, freeze-dried, and digested; a cadmium content of each tissue was determined by ICP-MS, and relative bioavailability was calculated; where the relative bioavailability was calculated according to the following formula:

$$Relative bioavailability = \frac{{( Liver + kidney + brain + femur )}_{Earthworm}}{Earthworm concentration} \times \frac{Positive control group concentration}{{( Liver + kidney + brain + femur )}_{positive}}$$

where, (liver+kidney+brain+femur)_{Earthworm} was the sum of cadmium content in the liver, kidney, brain and femur tissues of the mice determined 7 days after administration in the earthworm group (unit: μg/g dry weight), (liver+kidney+brain+femur)positive was the sum of cadmium content in the liver, kidney, brain and femur tissues of the mice determined 7 days after administration in the low-concentration positive control group, the medium-concentration positive control group, and the high-concentration positive control group (unit: μg/g dry weight).

A method for determining cadmium by ICP-MS included:

A single element standard solution of cadmium was precisely measured, and a series of solutions containing Cd at a concentration of 0.2, 0.5, 2, 4, and 10 μg/mL were prepared using a 5% HNO₃ solution, and a standard curve was drawn. The working conditions of ICP-MS were shown in Table 1.

TABLE 1
Working parameters of ICP-MS instrument

| | | | | Working | |
|---|---|---|---|---|---|
| Item | Working | | Item | parameter | |
| Plasma gas flow | 15.0 | L/min | Peristaltic pump | 0.20 | r/s |
| Sampling depth | 10 | mm | Number of | 1 | |
| | | | repetitions | | |
| Nebulization | 2° | C. | Auxiliary | 0.8 | L/min |
| chamber | | | gas flow | | |
| He gas flow | 5 | mL/min | Carrier | 0.8 | L/min |
| | | | gas flow | | |

| | | | | |
|---|---|---|---|---|
| Radio-frequency | 1550 | W | Data | Peak |
| power | | | sampling | hopping |
| | | | mode | sampling |

| | | | |
|---|---|---|---|
| Determination | 6 | Number | 100 |
| point/peak | | of scans | |
| Number of | 3 | | |
| repetitions | | | |

According to an analysis of fitting degree of the curve, a correlation coefficient of cadmium was determined to be 0.997, with a desirable linear relationship.

(2) Bioaccessibility of Cadmium in Earthworm Based on In Vitro PBET Method

1) Preparation of extracted gastric juice and investigation of a dissolution rate of cadmium in earthworm: Earthworm samples were crushed, sieved through a 50-mesh sieve to obtain earthworm powder. To 0.5 g of the earthworm powder was added 50 mL of simulated gastric juice to obtain a mixture. Argon was introduced for 1 min to 2 min to simulate an anaerobic environment in the gastrointestinal tract, the mixture was shaken at 100 r/min for 1 h in a constant-temperature water bath at 37° C., and centrifuged at 4,000 r/min for 5 min; 25 mL of a resulting supernatant was concentrated to 3 mL at low temperature on an electric hot plate, and 5 mL of nitric acid was added to the supernatant after cooling, followed by digestion in a microwave digestion apparatus; after cooling, a resulting digestion solution was diluted to a 50 mL volumetric flask with ultrapure water for testing; meanwhile, a reagent blank solution was prepared in the same way. The simulated gastric juice was prepared by mixing 1.25 g of pepsin, 0.5 g of sodium citrate, 0.5 g of sodium malate, 420 μL of lactic acid, and 500 μL of acetic acid, making up the volume to 1 L after dissolving, and adjusting pH to a value of 2.0 with hydrochloric acid.

2) Preparation of extracted intestinal juice and investigation of a dissolution rate of cadmium in earthworm: after gastric juice extraction, 50 mL of a simulated intestinal juice was added to residues to obtain a mixture, the mixture was shaken at 100 r/min for 4 h in a constant-temperature water bath at 37° C., and centrifuged at 4,000 r/min for 5 min; 25 mL of a resulting supernatant was concentrated to 3 mL at low temperature on an electric hot plate, and 5 mL of nitric acid was added to the supernatant after cooling, followed by digestion in a microwave digestion apparatus; after cooling, a resulting digestion solution was diluted and made up to 50 mL in a volumetric flask with ultrapure water for testing; meanwhile, a reagent blank solution was prepared in the same way. The simulated intestinal juice was prepared by mixing 0.5 g of a pancreatic enzyme and 1.75 g of ammonium cholate, making up the volume to 1 L after dissolving, and adjusting pH to a value of 7.0 with NaOH.

After the extraction, the residual cadmium was determined in the extracted gastric juice and intestinal juice using the ICP-MS method in step (1) separately to obtain the bioaccessibility of cadmium in earthworm.

(3) A correlation model was constructed according to results of the relative bioavailability and the bioaccessibility to obtain a regression equation.

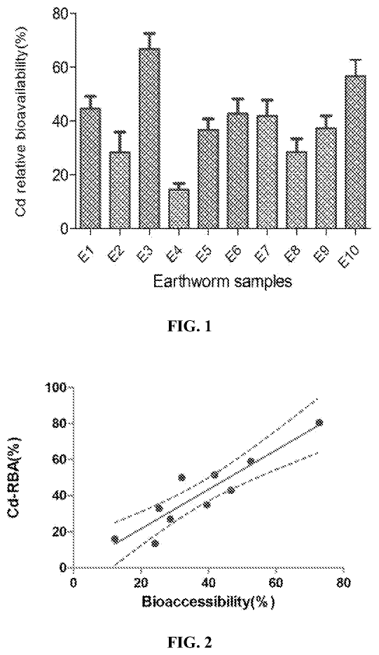

FIG. 1 shows relative bioavailability results of cadmium in 10 batches of earthworm determined in step (1).

The results in FIG. 1 show that the relative bioavailability of cadmium in 10 batches of earthworm is 13.5% to 80.5%.

FIG. 2 shows the correlation model of the bioavailability results and in vitro bioaccessibility results of cadmium in earthworm in step (3).

FIG. 2 shows that the regression equation is as follow: relative bioavailability=1.09×bioaccessibility+6.97, where r²=0.84.

### EXAMPLE 2

A method for constructing a correlation model between relative bioavailability and bioaccessibility of cadmium in earthworm included the following steps:

(1) Determination of Relative Bioavailability of Cadmium in Earthworm

After adaptive feeding, female mice having a body weight of 18 g to 22 g were randomly grouped (6 mice/group), and weighed after fasting overnight; 10 batches of earthworm groups were given multiple doses of intragastric administration of earthworm; high-concentration, medium-concentration and low-concentration positive control groups were administered with corresponding concentrations of cadmium by gavage, separately; meanwhile, mice administered with normal saline by gavage were used as a blank control group. The grouping and administration were conducted as follows:

1) control group: 6 mice were subjected to administration of normal saline at 1 mL/mice by gavage;, once a day;

2) low-concentration positive control group: 6 mice were subjected to administration at 1 mL/mice by gavage with a Cd concentration of 3.5mg/kg, once a day;

3) medium-concentration positive control group: 6 mice were subjected to administration at 1 mL/mice by gavage with a Cd concentration of 18mg/kg, once a day;

4) high-concentration positive control group: 6 mice were subjected to administration at 1 mL/mice by gavage with a Cd concentration of 95 mg/kg, once a day; and

5) earthworm group: 6 mice were subjected to administration at 1 mL/mice by gavage with a earthworm concentration of 1.128 mg/kg, once a day.

After 8 days of continuous administration, the mice were sacrificed, and liver, kidney, brain, and femur tissues of the mice were taken out, weighed, freeze-dried, and digested; a cadmium content of each tissue was determined by ICP-MS, and relative bioavailability was calculated; where the relative bioavailability was calculated according to the following formula:

$$Relative bioavailability = \frac{{( Liver + kidney + brain + femur )}_{Earthworm}}{Earthworm concentration} \times \frac{Positive control group concentration}{{( Liver + kidney + brain + femur )}_{positive}}$$

where, (liver+kidney+brain+femur)_{Pheretima} was the sum of cadmium content in the liver, kidney, brain and femur tissues of the mice determined 8 d after administration in the earthworm group (unit: μg/g dry weight), (liver+kidney+brain 30 femur)ₚₒₛᵢₜᵢᵥₑ was the sum of cadmium content in the liver, kidney, brain and femur tissues of the mice determined 8 days after administration in the low-concentration positive control group, the medium-concentration positive control group, and the high-concentration positive control group (unit: μg/g dry weight).

The method for determining cadmium by ICP-MS included:

A single element standard solution of cadmium was precisely measured, and a series of solutions containing Cd at a concentration of 0.2, 0.5, 2, 4, and 10 μg/mL were prepared using a 5% HNO₃ solution, and a standard curve was drawn. The working conditions of ICP-MS are shown in Table 1.

TABLE 1
Working parameters of ICP-MS instrument

| | | | | Working | |
|---|---|---|---|---|---|
| Item | Working | | Item | parameter | |
| Plasma gas flow | 15.0 | L/min | Peristaltic pump | 0.20 | r/s |
| Sampling depth | 10 | mm | Number of | 1 | |
| | | | repetitions | | |
| Nebulization | 2° | C. | Auxiliary | 0.8 | L/min |
| chamber | | | gas flow | | |
| He gas flow | 5 | mL/min | Carrier | 0.8 | L/min |
| | | | gas flow | | |

| | | | | |
|---|---|---|---|---|
| Radio- | 1550 | W | Data | Peak |
| frequency | | | sampling | hopping |
| power | | | mode | sampling |

| | | | |
|---|---|---|---|
| Determination | 6 | Number | 100 |
| point/peak | | of scans | |
| Number of | 3 | | |
| repetitions | | | |

According to an analysis of fitting degree of the curve, a correlation coefficient of cadmium was determined to be 0.997, with a desirable linear relationship.

(2) Bioaccessibility of Cadmium in Earthworm Based on In Vitro PBET Method

1) Preparation of extracted gastric juice and investigation of a dissolution rate of cadmium in earthworm: earthworm samples were crushed, sieved through a 50-mesh sieve to obtain earthworm powder. To 0.5 g of the earthworm powder was added 50 mL of simulated gastric juice to obtain a mixture. Argon was introduced for 1 min to 2 min to simulate an anaerobic environment in the gastrointestinal tract, the mixture was shaken at 100 r/min for 1 h in a constant-temperature water bath at 37° C., and centrifuged at 4,000 r/min for 5 min; 25 mL of a resulting supernatant was concentrated to 3 mL at low temperature on an electric hot plate, and 5 mL of nitric acid was added to the supernatant after cooling, followed by digestion in a microwave digestion apparatus. After cooling, a resulting digestion solution was made up to a 50 mL in a volumetric flask with ultrapure water for testing; meanwhile, a reagent blank solution was prepared in the same way. The simulated gastric juice was prepared by mixing 1.25 g of pepsin, 0.5 g of sodium citrate, 0.5 g of sodium malate, 420 μL of lactic acid, and 500 μL of acetic acid, making up to 1 L after dissolving, and adjusting pH to a value of 2.0 with hydrochloric acid.

2) Preparation of extracted intestinal juice and investigation of a dissolution rate of cadmium in earthworm: after gastric juice extraction, 50 mL of simulated intestinal juice was added to residues to obtain a mixture, the mixture was shaken at 100 r/min for 4 h in a constant-temperature water bath at 37° C., and centrifuged at 4,000 r/min for 5 min; 25 mL of a resulting supernatant was concentrated to 3 mL at low temperature on an electric hot plate, and 5 mL of nitric acid was added to the supernatant after cooling, followed by digestion in a microwave digestion apparatus; after cooling, a resulting digestion solution was diluted to a 50 mL in a volumetric flask with ultrapure water for testing; meanwhile, a reagent blank solution was prepared in the same way. The simulated intestinal juice was prepared by 0.5 g of a pancreatic enzyme and 1.75 g of ammonium cholate, making up to 1 L after dissolving, and adjusting pH to a value of 7.0 with NaOH.

After the extraction, the residual cadmium was determined in the extracted gastric juice and intestinal juice using the ICP-MS method in step (1) separately to obtain the bioaccessibility of cadmium in earthworm.

A correlation model was constructed according to results of the relative bioavailability and the bioaccessibility to obtain a regression equation.

### EXAMPLE 3

A method for constructing a correlation model between relative bioavailability and bioaccessibility of cadmium in earthworm included the following steps:

(1) Determination of Relative Bioavailability of Cadmium in Earthworm

After adaptive feeding, female mice having a body weight of 18 g to 22 g were randomly grouped (6 mice/group), and weighed after fasting overnight; 10 batches of earthworm groups were given multiple doses of intragastric administration of earthworm; high-concentration, medium-concentration and low-concentration positive control groups were administered with corresponding concentrations of cadmium by gavage, separately; meanwhile, mice administered with normal saline by gavage were used as a blank control group. The grouping and administration were conducted as follows:

1) control group: 6 mice were subjected to administration of normal saline at 1 mL/mice by gavage;, once a day;

2) low-concentration positive control group: 6 mice were subjected to administration at 1 mL/mice by gavage with a Cd concentration of 4.5 mg/kg, once a day;

3) medium-concentration positive control group: 6 mice were subjected to administration at 1 mL/mice by gavage with a Cd concentration of 22 mg/kg, once a day;

4) high-concentration positive control group: 6 mice were subjected to administration at 1 mL/mice by gavage with a Cd concentration of 105 mg/kg, once a day; and

5) earthworm group: 6 mice were subjected to administration at 1 mL/mice by gavage with a earthworm concentration of 7.401 mg/kg, once a day.

After 6 days of continuous administration, the mice were sacrificed, and liver, kidney, brain, and femur tissues of the mice were taken out, weighed, freeze-dried, and digested; cadmium content of each tissue was determined by ICP-MS, and relative bioavailability was calculated; where the relative bioavailability was calculated according to the following formula:

$$Relative bioavailability = \frac{{( Liver + kidney + brain + femur )}_{Earthworm}}{Earthworm concentration} \times \frac{Positive control group concentration}{{( Liver + kidney + brain + femur )}_{positive}}$$

where, (liver+kidney+brain+femur)_{earthworm} was the sum of cadmium content in the liver, kidney, brain and femur tissues of the mice determined 6 days after administration of the earthworm group (unit: μg/g dry weight), (liver+kidney+brain+femur)ₚₒₛᵢₜᵢᵥₑ was the sum of cadmium content in the liver, kidney, brain and femur tissues of the mice determined 6 days after administration of the low-concentration positive control group, the medium-concentration positive control group, and the high-concentration positive control group (unit: μg/g dry weight).

The method for determining cadmium by ICP-MS included:

A single element standard solution of cadmium was precisely measured, and a series of solutions containing Cd at a concentration of 0.2, 0.5, 2, 4, and 10 μg/mL were prepared using a 5% HNO₃ solution separately, and a standard curve was drawn. The working conditions of ICP-MS are shown in Table 1.

TABLE 1
Working parameters of ICP-MS instrument

| | | | | Working | |
|---|---|---|---|---|---|
| Item | Working | | Item | parameter | |
| Plasma gas flow | 15.0 | L/min | Peristaltic pump | 0.20 | r/s |
| Sampling depth | 10 | mm | Number of | 1 | |
| | | | repetitions | | |
| Nebulization | 2° | C. | Auxiliary | 0.8 | L/min |
| chamber | | | gas flow | | |
| He gas flow | 5 | mL/min | Carrier | 0.8 | L/min |
| | | | gas flow | | |

| | | | | |
|---|---|---|---|---|
| Radio- | 1550 | W | Data | Peak |
| frequency | | | sampling | hopping |
| power | | | mode | sampling |

| | | | |
|---|---|---|---|
| Determination | 6 | Number | 100 |
| point/peak | | of scans | |
| Number of | 3 | | |
| repetitions | | | |

According to analysis of the fitting degree of the curve, a correlation coefficient of cadmium was determined to be 0.997, with a desirable linear relationship.

(2) Bioaccessibility of Cadmium in Earthworm Based on In Vitro PBET Method

1) Preparation of extracted gastric juice and investigation of a dissolution rate of cadmium in earthworm: Earthworm samples were crushed, sieved through a 50-mesh sieve to obtain earthworm powder, 0.5 g of the earthworm powder was added with 50 mL of simulated gastric juice to obtain a mixture. Argon was introduced for 1 min to 2 min to simulate an anaerobic environment in the gastrointestinal tract, the mixture was shaken at 100 r/min for 1 h in a constant-temperature water bath at 37° C., and centrifuged at 4,000 r/min for 5 min; 25 mL of resulting supernatant was concentrated to 3 mL at low temperature on an electric hot plate, and 5 mL of nitric acid was added to the supernatant after cooling, followed by digestion in a microwave digestion apparatus; after cooling, a resulting digestion solution was diluted to a 50 mL in a volumetric flask with ultrapure water for testing; meanwhile, a reagent blank solution was prepared in the same way. The simulated gastric juice was prepared by mixing 1.25 g of pepsin, 0.5 g of sodium citrate, 0.5 g of sodium malate, 420 μL of lactic acid, and 500 μL of acetic acid, making up to 1 L after dissolving, and adjusting pH to a value of 2.0 with hydrochloric acid.

2) Preparation of extracted intestinal juice and investigation of a dissolution rate of cadmium in earthworm: after gastric juice extraction, 50 mL of a simulated intestinal juice was added to residues to obtain a mixture, the mixture was shaken at 100 r/min for 4 h in a constant-temperature water bath at 37° C., and centrifuged at 4,000 r/min for 5 min; 25 mL of a resulting supernatant was concentrated to 3 mL at low temperature on an electric hot plate, and 5 mL of nitric acid was added to the supernatant after cooling, followed by digestion in a microwave digestion apparatus; after cooling, a resulting digestion solution was diluted to a 50 mL in a volumetric flask with ultrapure water for testing; meanwhile, a reagent blank solution was prepared in the same way. The simulated intestinal juice was prepared by mixing 0.5 g of a pancreatic enzyme and 1.75 g of ammonium cholate, making up to 1 L after dissolving, and adjusting pH to a value of 7.0 with NaOH.

After the extraction, the residual cadmium was determined in the extracted gastric juice and intestinal juice using the ICP-MS method in step (1) separately to obtain the bioaccessibility of cadmium in earthworm.

A correlation model was constructed according to results of the relative bioavailability and the bioaccessibility to obtain a regression equation.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1. A method for constructing a correlation model between relative bioavailability and bioaccessibility of cadmium in earthworm, comprising the following steps:
grouping mice, conducting successive administration for 6 days to 8 days, and sacrificing the mice; determining a cadmium content in liver, kidney, brain, and femur tissues of the mice, and calculating relative bioavailability of cadmium in earthworm;
simulating gastrointestinal digestion by a physiologically-based extraction test (PBET) method and extracting digestive juices, and determining a residual amount of cadmium in gastric juice and intestinal juice separately to obtain bioaccessibility of cadmium in the earthworm; and
constructing a correlation model according to the relative bioavailability and the bioaccessibility of cadmium in the earthworm to obtain a regression equation.

2. The method according to claim 1, wherein the grouping is conducted specifically as follows: the mice fed with normal saline are used as a blank control group, the mice fed with cadmium are used as a positive control group, and the mice fed with the earthworm are used as an earthworm group; and the positive control group is divided into a low-concentration positive control group, a medium-concentration positive control group, and a high-concentration positive control group.

3. The method according to claim 2, wherein the low-concentration positive control group has a cadmium concentration of 3.5 mg/kg to 4.5 mg/kg; the medium-concentration positive control group has a cadmium concentration of 18 mg/kg to 22 mg/kg; the high-concentration positive control group has a cadmium concentration of 95 mg/kg to 105 mg/kg; and the earthworm group has an earthworm concentration of 1.128 mg/kg to 7.401 mg/kg.

4. The method according to claim 1, wherein a calculation formula of the relative bioavailability is as follows:
$$Relative bioavailability = \frac{{( Liver + kidney + brain + femur )}_{Earthworm}}{Earthworm concentration} \times \frac{Positive control group concentration}{{( Liver + kidney + brain + femur )}_{positive}} ;$$
wherein (liver+kidney+brain+femur)_{Earthworm} is the sum of cadmium content in the liver, kidney, brain and femur tissues of the mice determined 6 days to 8 days after administration in the earthworm group, (liver+kidney+brain+femur)ₚₒₛᵢₜᵢᵥₑ is the sum of cadmium content in the liver, kidney, brain and femur tissues of the mice determined 6 days to 8 days after administration in the low-concentration positive control group, the medium-concentration positive control group, and the high-concentration positive control group.

5. The method according to claim 1, wherein cadmium content in the tissues is determined by an inductively coupled plasma-mass spectrometry (ICP-MS) method; and the residual amount of cadmium in the gastric juice and the intestinal juice is determined by the ICP-MS method.

6. The method according to claim 1, wherein the administration is conducted by gavage.

7. The method according to claim 5, wherein working parameters of the ICP-MS method are as follows.

| | | | | Working | |
|---|---|---|---|---|---|
| Item | Working | | Item | parameter | |
| Plasma gas flow | 15.0 | L/min | Peristaltic pump | 0.20 | r/s |
| Sampling depth | 10 | mm | Number of | 1 | |
| Nebulization | 2° | C. | Auxiliary | 0.8 | L/min |
| chamber | | | gas flow | | |
| He gas flow | 5 | mL/min | Carrier | 0.8 | L/min |
| | | | gas flow | | |
| | | | | |
|---|---|---|---|---|
| Radio- | 1550 | W | Data | Peak |
| frequency | | | sampling | hopping |
| power | | | mode | sampling |
| | | | |
|---|---|---|---|
| Determination | 6 | Number | 100 |
| point/peak | | of scans | |
| Number of | 3 | | |
| repetitions | | | |

8. The method according to claim 1, wherein the regression equation is as follows:
relative bioavailability=1.09×bioaccessibility+6.97.

9. The method according to claim 6, wherein working parameters of the ICP-MS method comprise:
a plasma gas flow is about 15.0 L/min;
a sampling depth is about 10 mm;
a nebulization chamber is at about 2° C.;
an He gas flow is about 5 mL/min;
a radio-frequency power is about 1550 W;
a determination point/peak is about 6;
a number of repetitions is about 3;
a peristaltic pump is about 0.20 r/s;
a second number of repetitions is about 1;
an auxiliary gas flow is about 0.8 L/min;
a carrier gas flow is about 0.8 L/min;
a data sampling mode comprises peak hopping sampling; and
a number of scans is about 100 times.
